(19) United States
(12) Patent Application Publication
(10) Pub. No.: US 2008/0292610 A1
(43) Pub. Date: Nov. 27, 2008

(54) Medicaments containing enzymes from ciliates for promoting digestion in digestive disorders

(75) Inventor: Marcus Hartmann, Munster (DE)

Correspondence Address: JACOBSON HOLMAN PLLC, 400 SEVENTH STREET N.W., SUITE 600, WASHINGTON, DC, 20004, US

(73) Assignee: Cilian AG

(21) Appl. No.: 11/878,618

(22) Filed: Jul. 25, 2007

Related U.S. Application Data
(63) Continuation of application No. 10/399,551, filed on Jul. 23, 2003, now abandoned.

(30) Foreign Application Priority Data
Nov. 2, 2000 (DE) 100 54 239.5

(51) Int. Cl. A61K 38/43; A61P 43/00

(52) U.S. Cl. 424/94.63; 424/94.6

(57) ABSTRACT

A medicament containing enzymes from ciliates selected from the group consisting of hydrolases, lipases, proteases, amylases, glycosidases, phospholipases, phosphodiesterases, phosphatases.

The invention relates to a medicament containing enzymes from ciliates for treating digestive disorders.

Digestive disorders play an increasingly greater role in the general medical and internal medical practice. Such digestive disorders are in many cases the consequence of a more or less pronounced deficiency in so-called pancreatic enzymes. In a healthy state, these enzymes are synthesized in the pancreas by highly specialized cells, the so-called acinic cells, and secreted by exocytosis through juice glands and the main pancreatic duct into the duodenum. The daily amount of pancreatic secretion is about 2 liters. In addition to fat-digesting lipase, the pancreatic secretion also contains enzymes for the digestion of proteins (trypsin, chymotrypsin and carboxypeptidases) and carbohydrates (α-amylase). The secretion of pancreatic enzymes is exactly controlled by endogenous control mechanisms by means of hormones, such as gastrin, secretin and pancreozymin. This control system can be disturbed by a large number of causes to result in a reduction of pancreatic enzyme secretion or in a complete subsiding of the exocrine pancreatic function. This in turn causes that the chyme is not digested in the small intestine, and a digestive disorder occurs. This disease of the digestive tract, which is also referred to as exocrine pancreatic insufficiency, can have different causes. In addition to dyspepsia caused by medicaments, chronic atrophic gastritis and chronic pancreatitis, frequently caused by alcohol consumption, disorders caused by surgery (e.g., Billroth I and II, vagotomy, pancreas resection) and cystic fibrosis are etiologic factors of pancreatic insufficiency. At any rate, chronic digestive disorders are of considerable social-medical and thus economic importance, because the symptoms frequently cause the patients to be nondescript and have a shortened expectation of life.

Pancreatogenic digestive disorders cause a lot of complaints in the patients, such as diarrhea, mass stools, sensations of repletion, upper abdominal complaints, weight loss etc.

Irrespective of the causes and the manifestation of pancreatogenic digestive disorders or pancreatic insufficiency, a substitution therapy with enzymes is always necessary for relieving the digestive disorders. This means that the lacking enzymes, predominantly lipase, protease and amylase, but also other enzymes, must be supplied externally. In the therapy, the enzymes are taken in orally by the patient mostly in the middle of the meal and go through the stomach and arrive in the small intestine, where they perform digestion of the chyme and thus adopt the function of the lacking endogenous pancreatic enzymes. The preparations employed must contain a sufficient amount of enzymes. In addition, the enzymes must be provided in an enteric formulation, have a small particle size and be completely bioavailable in the digestive tract.

For treating digestive disorders based on the lacking of pancreatic enzymes, especially the leading enzyme lipase and the protease, a wide variety of enzyme preparations are already on the market. These are partly based on pancreatic enzymes from pigs, such as the preparations Combizym®, Festal®, Pankreon®, Kreon®, Panzytrat®, Meteozym® or Enzym-Lefax N®, or on gastric enzymes, such as Citrapepsin®. In part, the preparations may also contain enzymes from mold extracts, such as Combizym® and Festal®. Further, the use of enzymes from fish or other marine animals is generally described (FR No. 1015566), as well as compositions of enzymes from the gastro-intestinal tract of krill (crustaceans from the class Euphausiaceae) and capelin fish (U.S. Pat. No. 4,695,457). Preparations containing pancreatic enzymes are mostly obtained from the slaughterhouse waste, for example, pancreas, of pigs. The final product of the preparation process is pancreatin. Pancreatin is a homogenizate from the cells of pancreatic tissues (usually from pigs). Due to the rupture of a large number of acinic cells, it contains, in addition to pancreatic enzymes, a wide variety of other enzymes and proteins as well as further high and low molecular weight compounds. The composition of pancreatin is due to its industrial preparation process. To obtain pancreatin, pancreas of pigs are deep-frozen as quickly as possible after slaughtering, collected and broken up mechanically. For the stabilization and activation of the enzymes, various additives are added to the homogenizate. This is followed by defatting with organic solvents, such as acetone, the removal of fibrous substances, and dewatering and drying by lyophilization. For the preparation of particular dosage forms, further galenic processing may be effected into micropellets, tablets, capsules, pastes, creams, gels, oils or other formulations. Frequently, pancreatin is mixed with various support materials and buffer substances. Further, granulated pancreatin is coated with acid-stable films or lacquers for protection against the low pH value of human gastric juice. The two latter processing steps are to ensure that the acid-labile pancreatic enzymes can fulfill their digestive function at the target site, the duodenum (small intestine).

In addition to the usual preparation of pancreatin, the lipase content in the final product can be increased by a successive extraction with chloroform, butanol and acetone. In a similar way as with pancreatin, enzyme compositions are obtained from the krill species Euphausia suberba and from the intestines of capelin fish (see U.S. Pat. No. 4,695,457). In this case too, a homogenizate is first prepared from the tissue and then further purified by centrifugation, extraction with chloroform, lyophilization and chromatographic steps. In each case, the object of the processing is as high as possible a content of pancreatic enzymes, such as lipase, protease and α-amylase. Further, the enzyme composition is to be as resistant to gastric juice as possible for the treatment of digestive disorders. In addition, the enzymes are to be released as quickly as possible in the digestive tract and especially in the duodenum and display their physiological activity. To avoid allergies, the enzyme composition should be free from ineffective proteins if possible, or have a high degree of purity. In addition, the preparation process should be inexpensive under pharmaeconomical aspects.

With pancreatin, a further purification by chromatographic methods is usually not effected, so that the desired enzymes are obtained in a neither purified nor homogeneous state. A disadvantage of such protein mixtures from cell homogenizates is the fact that they contain a wide variety of proteins from intracellular cell compartments (cytosol, nucleus, membranes of the organelles) of the pig pancreatic cells. These have no or no desired enzymatic activity and thus decrease the amount of active substance per dosage form supplied. The same holds if concentrated cell homogenizates from krill or the gastro-intestinal tract of capelin fish are obtained.

Another drawback of the preparation of enzymes and enzyme compositions from slaughterhouse waste (pancreas, gastro-intestinal tract of fish) and krill is the discontinuous process mode of the recovery. Usually, the organs (pancreas) are comminuted and homogenized in different steps. The homogenizate is subsequently defatted and dried. These steps of enzyme recovery cannot be operated continuously, since a considerable solids content is always employed, which precludes continuous further processing due to extraction and centrifugation steps. However, for the recovery of enzymes, continuous or semicontinuous production methods are optimal since the space time yields are higher and the costs are thus lower in such processes. However, the continuous recovery of enzymes requires that they are in an extracellular and dissolved form and need not be first released by the comminuting or homogenization of cells. Therefore, the pancreatin production process is not suitable for the continuous recovery of enzymes.

Another disadvantage of the enzymes from pancreas is their acid lability. Pancreatic enzymes are neutral or alkaline hydrolases. This means that on the one hand, they have their activity maximum between pH 7 and pH 8, and have a highly reduced activity under acidic conditions. On the other hand, low pH values inactivate their catalytic function by reversible or irreversible denaturing. For this reason, enzymes obtained from pancreas (pancreatin) must be protected from the low pH value of the gastric juice during the passage through the stomach by special methods of encapsulation or the addition of buffer substances. Such methods also increase the costs of medicaments which are based on the activity of pancreatin.

In addition, for particular groups of patients, a disadvantage of the use of pancreatic enzymes is the origin of pancreatin. Usually, the pancreas of pigs are used, which cannot be tolerated by patients of Judaic or Islamic religion due to religious instructions.

Finally, pancreatic enzymes from pigs cannot be employed with patients suffering from digestive disorders who have a pig protein allergy. In addition, pigs are considered a natural reservoir of human-pathogenic influenza viruses, so that contamination of pancreatin with such viruses cannot be rules out.

Therefore, it is the object of the invention to provide enzymes or enzyme compositions for medicaments which:
1) are in an extracellular state and can be obtained and purified from cell-free supernatant without homogenization (disruption) of tissues or cells;
2) can be obtained continuously in a biotechnological process of a harmless microorganism which is not genetically engineered;
3) are acid hydrolases, i.e., have their activity maximum at an acidic pH value and are more acid-stable than enzymes from pancreas;
4) are not derived from tissues or organs of pigs.

This object is achieved by a medicament containing enzymes selected from the group consisting of hydrolases, lipases, proteases, amylases, glycosidases, phospholipases, phosphodiesterases, phosphatases, and obtained from ciliates.

In particular, the invention relates to medicaments which are employed for promoting the digestion and for the treatment of digestive disorders.

Preferably, the medicaments according to the invention contain enzymes which are employed for digesting the macromolecules contained in foods, such as proteins, nucleic acids and carbohydrates, as well as other components of foods, such as fats or phospholipids, in the gastro-intestinal tracts of humans or animals.

The skilled person understands that enzymes from ciliates which do not belong to the previously employed lipases, proteases or amylases can also be employed for the promotion of digestion or for the treatment of digestive disorders according to the invention, since other enzymes can also promote the digestion in the gastrointestinal tract by the catalytic cleavage of food components.

In one embodiment of the invention, the enzymes have a pH optimum at pH 4-6.

The medicaments according to the invention preferably contain enzymes which are derives from ciliates of the genera Tetrahymena, Colpidium and Paramecium.

Preferably, the medicaments according to the invention contain pharmaceutically safe auxiliary agents and carriers.

The medicaments according to the invention are employed, in particular, in the form of tablets, micropellets, oils, juices, gels, suppositories, capsules, coated tablets.

The invention also relates to the use of enzymes from ciliates selected from the group consisting of hydrolases, lipases, proteases, amylases, glycosidases, phospholipases, phosphodiesterases and/or phosphatases for the preparation of a medicament for treating digestive disorders, especially dyspepsia caused by medicaments, chronic atrophic gastritis, chronic pancreatitis, acute pancreatitis, digestive disorder (maldigestion) caused by surgery, or one caused by cystic fibrosis.

The enzymes produced by protozoan of the order of ciliates and employed in the medicaments according to the invention are very suitable for the treatment of digestive disorders. In addition, enzymes and enzyme compositions from ciliates which are contained in the medicaments according to the invention as well as their preparation do not have the disadvantages of the above mentioned pancreatic enzymes or enzymes from capelin fish or krill.

Enzymes are released by ciliates into the surrounding culture medium. For example, Table 1 shows enzyme activities of different enzymes in the culture medium of ciliates. The enzyme activities represented in Table 1 were determined with the usual methods described in the literature. For measuring the lipase, an azo-casein test was used (Muricane, 1986). The determinations of the lipase and amylase were performed by analogy with the method prescriptions of the FIP for fungal amylase and microbial lipase (Demeester et al., in “Pharmaceutical Enzymes”, A. Lauwers and S. Scharpé [editors], Marcel Dekker, New York, 1997, pp. 372-382). For the determination of acid phosphatase and β-hexosamidinase, a p-nitrophenyl phosphate substrate and a p-nitrophenyl-N-acetyl-β-D-glucosamine substrate were employed, respectively, as described by Kiy et al. (1996). The phospholipase A₁ activity was determined with a radiometric enzyme test (Hartmann et al., 2000).

TABLE 1
Enzyme activity in the extracellular culture medium (U/I) after 72 h of
culturing on skim milk medium in a 2 liter fermenter

| | | | | β-hexos- | phospho- | acid |
|---|---|---|---|---|---|---|
| Ciliate | Protease | Lipase | α-amylase | aminidase | lipase A₁ | phosphatase |
| Tetrahymena | 800 U/I | 164 U/I | 20 U/I | 500 U/I | 10 U/I | 1000 U/I |
| Colpidium | 12 U/I | — | — | 40 U/I | 1.5 U/I | 80 U/I |

The ciliates which release enzymes into the culture medium can be fermented at low cost on inexpensive fermentations media at a high cell density and continuously. The enzymes can be filtered off cell-free from the fermenter through a perfusion module (microfilter) and thus be continuously removed from the fermentation medium. The fermentation process can be maintained over extended periods of time by a continuous supply of inexpensive nutritive media (components: skim milk medium and yeast extract).

### EXAMPLE 1

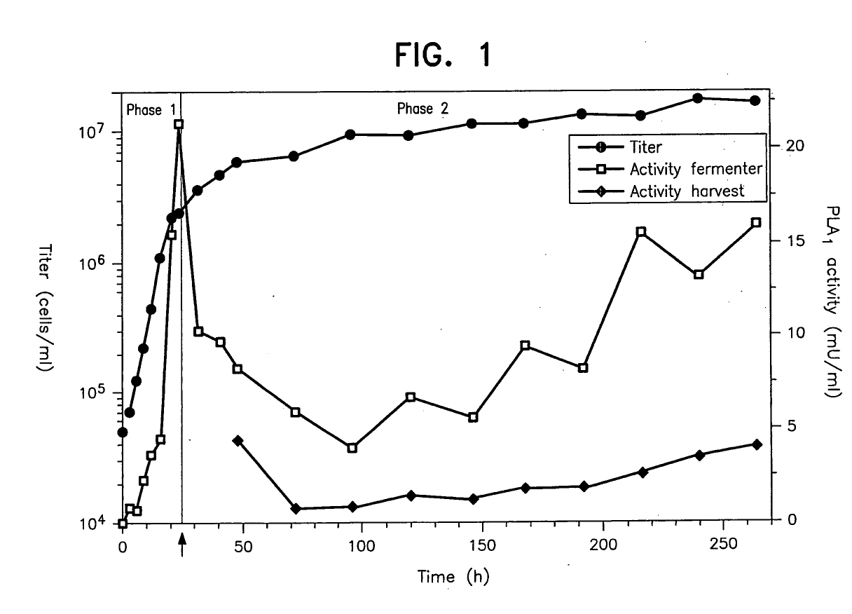

FIG. 1 shows the secretion kinetics of a ciliate, represented over a fermentation period of 14 days. For continuous fermentation with a perfusion module, the following procedure was employed:

The bioreactor system is based on a processor-controlled 2 liter fermenter (Biostat MD, Braun Diessel Biotech, Melsungen, Germany) with a digital DCU control unit and a pump unit. The harvesting of the cell-free supernatant was effected through a perfusion module, and a paddle impeller was used as a stirrer. A silicone oil concentration of 1 ml/l was used. The revolutions per minute of the stirrer was limited to 800 rpm for preventing damage to the cells. The concentration of the dissolved oxygen was kept constant at 60% by means of a cascade regulation. The aeration rate was selected as a control quantity of first priority, and the revolutions per minute of the stirrer was selected as a consecutive control of second priority. The measurement of the oxygen concentration was effected by means of an amperemetric O₂ electrode (Ingold Messtechnik, Steinbach). The temperature in the fermenter was kept at 30 by the double-walled vessel and a thermostat, and the pH regulation to pH 7 was effected during the continuous fermentation phase through a DCU-controlled correction agent pump using 4 M acetic acid. In the continuous fermentation phase, skim milk medium was supplied to the fermenter, and the consumed medium containing the enzymes was removed from the fermentation process. By using a pump which was controlled over a conductivity probe, the working volume could be kept constant at two liters. The cell-free supernatant was harvested particle-free through a perfusion module having a membrane pore size of about 0.3 μm. The perfusion module consisted of an S6/2 polypropylene capillary (Enka, Wuppertal) wound on a support and having outer and inner diameters of 2 and 1 mm, respectively, and a length of 2.8 m. The exchange of the medium volume per unit time was defined as the perfusion rate. The supply rate of the skim milk medium could be controlled through the number of revolutions of a peristaltic pump in such a way that a perfusion rate of one fermenter volume (two liters) per day was adjusted. Prior to autoclaving, the fermenter was completely mounted with the foam trap and charged with 1.8 l of skim milk medium without glucose. After autoclaving, 200 ml of 10% glucose solution was pumped in through the inlet. The connecting of the medium and harvest jars was effected through quick couplings in a sterile cabinet. In the sterile cabinet, the inoculation culture was transferred into a 500 ml Erlenmeyer flask with a bottom drain and pumped into the bioreactor through a flexible tube and a separate inoculation piece.

The ciliates remain undamaged in the fermentation, so that the culture medium only contains the proteins secreted by the ciliates and no intracellular components. For this reason, the enzymes from the fermentation or culture medium can be brought to high purity in few chromatographic purification steps.

### EXAMPLE 2

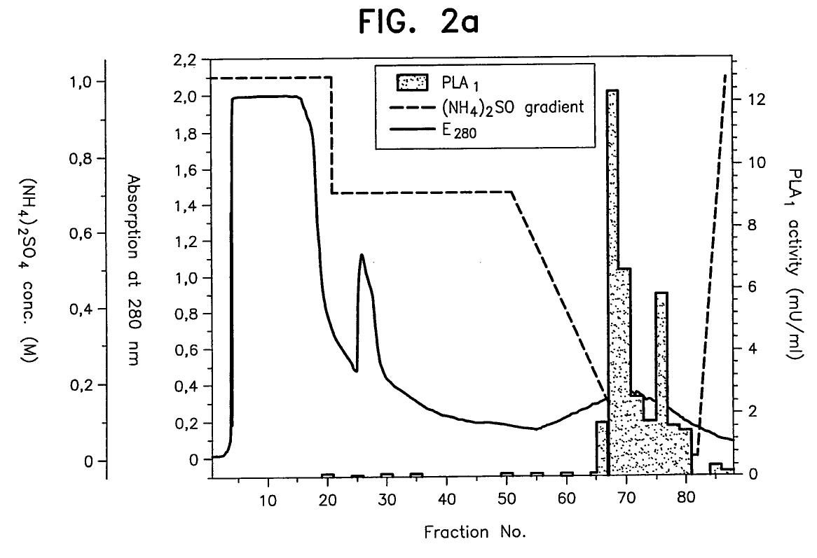

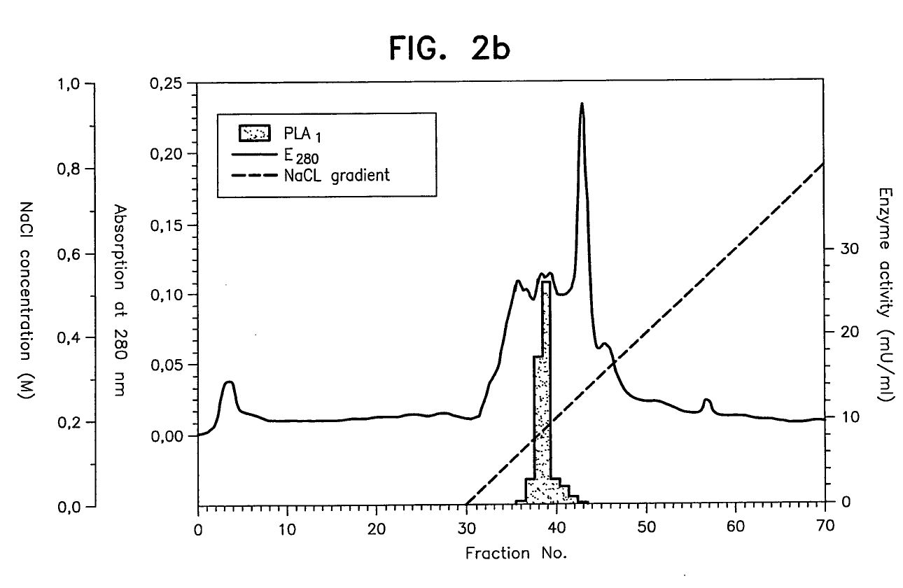

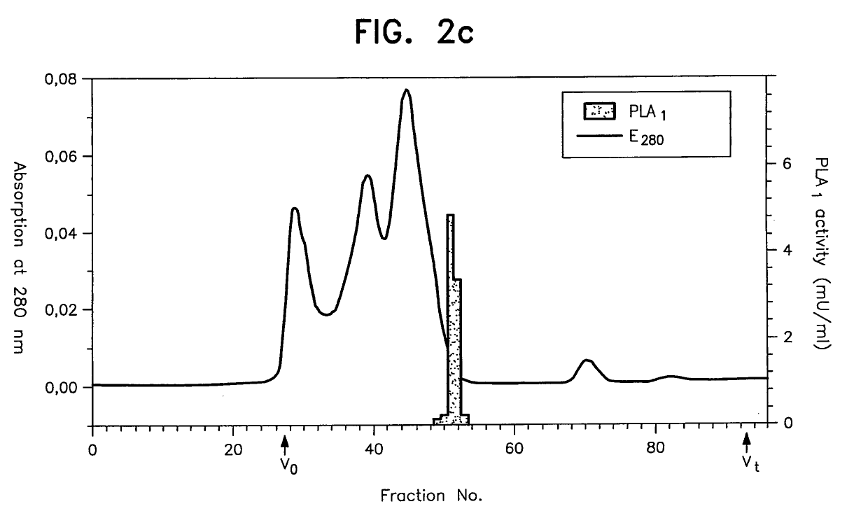

FIG. 2 shows column-chromatographic steps for the purification of a phospholipase from the culture medium of the ciliate Tetrahymena by way of example. The elution profiles of three successive column-chromatographic steps are shown. In detail, FIG. 2a shows the elution profile of hydrophobic interaction chromatography as step 1, FIG. 2b shows the elution profile of anion-exchange chromatography as step 2, and FIG. 2c shows the elution profile of size exclusion chromatography as step 3. For the subsequent chromatographies, the active fractions of the respectively previous chromatography were used. Thus, the enzyme could be purified to almost complete homogeneity after the last step (no foreign activities, low content of foreign proteins). By analogy with this purification scheme, other enzymes from ciliates, such as protease, lipase, amylase or β-hexosaminidase, may also be purified.

Except for one facultatively pathogenic representative (Balantidium coli), ciliates are free-living (non-parasitic) apathogenic microorganisms. Thus, the GRAS status (“generally recognized as safe”) for the ciliate Tetrahymena, for example, is stated in the literature (Tiedtke, 1994). In addition, it is considered certain that ciliates do not harbor any endoparasites which can be transferred to other organisms. In addition, for the ciliate species important to biotechnology, such as Tetrahymena or Colpidium, no viruses or other endoparasites exist. Therefore, contamination of the enzyme composition with toxic or pyrogenic impurities can be excluded.

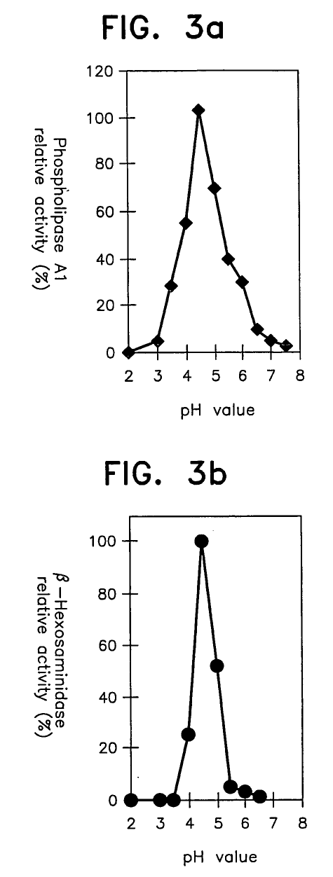

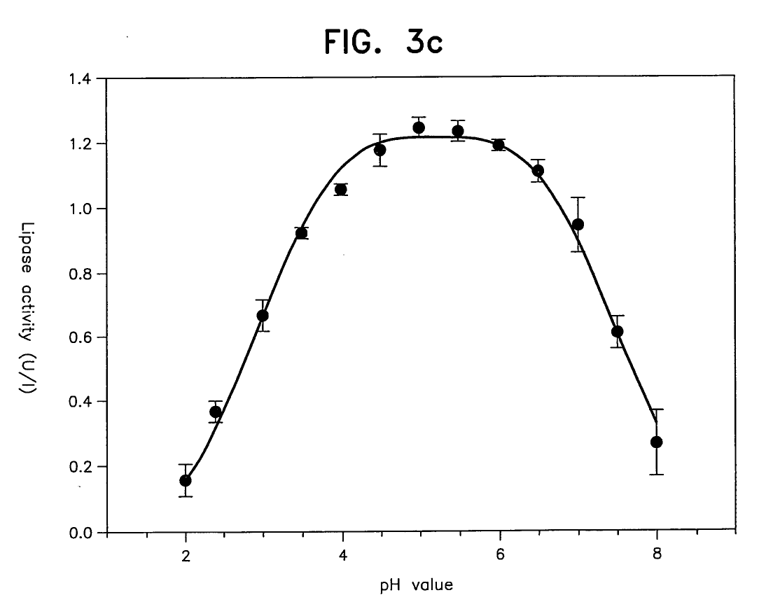

FIG. 3 shows the representation of the relative enzyme activities of 3 enzymes from the culture medium of the ciliate Tetrahymena at different pH values (a-phospholipase A₁, b-triacylglycerol-lipase, c-β-hexosaminidase).

The enzymes from ciliates, being acid hydrolases, have an acidic pH optimum. FIG. 3 shows the enzyme activities of 3 enzymes from the culture medium of the ciliate Tetrahymena at different pH values. In detail, the relative enzyme activities of phospholipase A₁ (FIG. 3a) and of β-hexosaminidase (FIG. 3b) and the absolute enzyme activity of lipase (FIG. 3c) are represented.

It becomes clear that the pH optimum for enzymes from ciliates is between pH 4.1 and 6.5. The protease from ciliates shows a high enzymatic activity even at a pH value of as low as 3. In the following. Table 2, the activity of the ciliate protease from the cell-free supernatant (culture medium) is shown as a function of the pH value. As already described above, the enzyme activities were determined by analogy with the method prescriptions of the FIP for fungal amylase and microbial lipase.

TABLE 2

| pH value | 3 | 4 | 5 | 6 |
|---|---|---|---|---|
| Protease | 2266 ± 19% | 1854 ± 12% | 1483.2 ± 11% | 11948 ± 25% |
| activity | | | | |
| (units/ | | | | |
| liter) | | | | |

For this reason, ciliate enzymes are also more stable towards acids as compared to pancreatic enzymes. Consequently, they have a considerably higher activity in the duodenum after a passage through the stomach as compared to pancreatic enzymes.

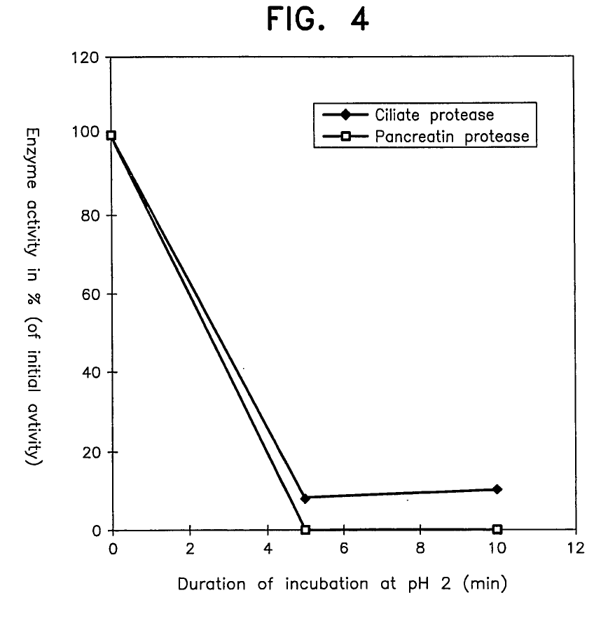

FIG. 4 shows the acid stability of a protease from the ciliate Tetrahymena as compared to pancreatin for a 10 minutes phase of action at a typical pH value as found in the gastric juice (pH 1.5). The low pH value was simulated by the action of a high molarity acidic buffer (1 M glycine/HCl, pH 1.5) at 37.

1-7. (canceled)

8. A composition useful as a medicament comprising lipase and protease enzymes isolated from ciliates.

9. The composition according to claim 8, characterized in that the enzymes have optimum activity at pH 4-6.

10. The composition according to claim 8, characterized in that the ciliates are selected from the group consisting of the genera Tetrahymena, Colpidium, and Paramecium.

11. The composition according to claim 8, characterized by being in a form selected from the group consisting of a tablet, micropellet, oil, juice, gel, suppository, capsule, and coated tablet.

12. The composition according to claim 8 further comprising a pharmaceutically acceptable carrier or diluent.

13. The composition according to claim 12, characterized in that the enzymes have optimum activity at pH 4-6.

14. The composition according to claim 12, characterized in that the ciliates are selected from the group consisting of the genera Tetrahymena, Colpidium, and Paramecium.

15. The composition according to claim 12, characterized by being in a form selected from the group consisting of a tablet, micropellet, oil, juice, gel, suppository, capsule, and coated tablet.

16. A method of treating a digestive disorder comprising administering an effective amount of the composition of claim 8 to a patient in need thereof.

17. A method of treating a digestive disorder comprising administering an effective amount of the composition of claim 9 to a patient in need thereof.

18. A method of treating a digestive disorder comprising administering an effective amount of the composition of claim 10 to a patient in need thereof.

19. A method of treating a digestive disorder comprising administering an effective amount of the composition of claim 11 to a patient in need thereof.

20. A process of preparing a composition useful as a medicament comprising the sequential steps of:
a) fermenting ciliates in a culture medium to secrete lipase and protease enzymes,
b) removing cell-free medium containing the secreted lipase and protease enzymes,
c) purifying the secreted lipase and protease enzymes, and
d) combining the purified lipase and protease enzymes with a pharmaceutically acceptable carrier or diluent.

21. The process according to claim 20, characterized in that the ciliates are selected from the group consisting of the genera Tetrahymena, Colpidium, and Paramecium.
